(19) United States
(12) Patent Application Publication
(10) Pub. No.: US 2008/0208152 A1
(43) Pub. Date: Aug. 28, 2008

(54) Incontinence Article

(75) Inventors: Axel Eckstein, Heidenheim (DE); Rudolf Groener, Soehnstetten (DE); Ruediger Kesselmeier, Herbrechtingen (DE); Christian Koch, Bachhagel (DE)

Correspondence Address: YOUNG & BASILE, P.C., 3001 WEST BIG BEAVER ROAD, SUITE 624, TROY, MI, 48084, US

(73) Assignee: PAUL HARTMANN AG, Heidenheim (DE)

(21) Appl. No.: 11/996,195

(22) PCT Filed: Jul. 8, 2006
(86) PCT No.: PCT/EP06/06697

§ 371 Date: Jan. 18, 2008

(30) Foreign Application Priority Data
Jul. 29, 2005 (DE) 10 2005 035 544.7

(51) Int. Cl. A61F 13/15

(52) U.S. Cl. 604/365; 604/385.03; 604/387; 604/389; 604/385.01

(57) ABSTRACT

An absorbent incontinence product with a main piece formed of a front section, a back section and a crotch section which lies in a longitudinal direction between the front and the back sections and is intended to be placed between the legs of a user. The main part has an absorbent body and first material sections which are attached to the back section and/or to the front section and extend in the transverse direction over first longitudinal side edges of the main piece and second material sections which are attached to the back section and/or to the front section and extend in the transverse direction over second longitudinal side edges of the main part. The first and second material sections connect the front section to the back section when the product is applied. The material sections, prior to use of the folded product, are each folded inwards towards the side of the main piece facing the body about a fold axis extending in the longitudinal direction to form an arrangement that is folded over on itself so that the second material section comes to lie at least partly beneath the first material section and the folded arrangement is detachably secured at a first joining point.

### BACKGROUND

The present disclosure relates to an absorbent incontinence article, specifically for incontinent adults.

In the case of incontinence articles, having a main piece consisting of a front section, a back section and a crotch section lying in the longitudinal direction which is positioned between the legs of a user, the main piece comprising an absorbent body, and having first and second material segments adjoining the back section and/or the front section which extend in the transverse, or circumferential hip, direction beyond longitudinal side edges of the main piece and connect the front section to the back section when the article is worn, the material segments, particularly material segments adjoining only the back section, can be formed from a different material than the main piece or a component of the main piece, for example a fluid-impermeable backsheet or a fluid-permeable topsheet. For example, the material segments forming the side parts, frequently referred to as the “wings” of the incontinence article, can be configured to breathe, specifically to be permeable to air or vapor, whereas the main piece, frequently referred to as the chassis, can be configured to be fluid-impermeable, specifically moisture-impermeable. To close the incontinence article, the material segments forming the side parts which are preferably joined non-releasably to the back section are brought to the stomach side of the user and releasably attached to the outside of the front section of the main piece facing away from the body. Closing elements on the side parts of the incontinence article operating mechanically or adhesively are frequently used which then act in concert with suitably configured landing zones in the front section of the main piece.

EP 1 269 949 A2 does not show an incontinence article in which the side parts projecting from a back section are fastened to the front section, but instead a so-called belt diaper in which the side parts projecting from the back section are joined together to form a hip opening continuously closed in the circumferential direction. The side parts projecting laterally from the back section which form the hip belt are Z-folded on themselves and secured in this configuration, specifically by an adhesive which loses adhesive strength immediately after the diaper is manufactured.

It would be desirable to provide an absorbent incontinence article of the type described initially as its point of departure with relatively wide material segments joined to the main piece to improve the handling of these material segments during production in a high-speed machine and to make handling the incontinence article during use by the wearer or by health-care staff as user-friendly as possible while protecting them from soiling.

### SUMMARY

Prior to the use of the folded incontinence article, the material segments are folded inward about a fold axis ruining in the longitudinal direction onto the side of the main piece facing the body to create an arrangement folded on itself such that the second material segment comes to lie at least partially under the first material segment and this folded arrangement is releasably attached at a first joining point.

While the hip belt is configured relatively narrow in the case of belt diapers, absorbent incontinence articles of the type described at the beginning having fairly wide lateral material segments are desirable. Such an incontinence article can be created in which the arrangement of the material segments folded over on themselves is releasably attached so that it is retained in the folded arrangement in the high-speed machine and the material segments cannot flutter. Attaching the folded arrangement of the two material segments releasably also proves advantageous during removal from the packaging and during preparations to apply the hygiene article, since the folded arrangement represents secure protection from contamination of the side facing the body, specifically of the top sheet of the incontinence article.

### BRIEF DESCRIPTION OF THE DRAWINGS

Additional features, details and advantages of the incontinence article become more apparent from the attached patent claims, the drawings and subsequent description of a preferred embodiment of the invention. In the drawing:

FIG. 1a shows a schematic representation of a material segment clamped in a tensile test apparatus;

FIG. 1b shows a not-to-scale representation of a material segment in the configuration folded on itself;

FIG. 1c shows a schematic representation of a back section of a hygiene article clamped in a tensile test machine with two accordion-folded material segments folded on themselves and folded inwards into an arrangement;

FIG. 1d shows a not-to-scale representation of a separated back section of a hygiene article with two accordion-folded material segments, folded on themselves and folded inward to form a folded arrangement;

FIGS. 2 and 3 show force-displacement plots determined in a tensile test;

FIG. 4 shows a plan view of a hygiene article in a schematic representation;

FIG. 5 shows a sectional view of the hygiene article from line A-A in FIG. 4;

FIG. 6a shows a not-to-scale sectional view with two accordion-folded material segments from line B-B in FIG. 6b folded on themselves and folded inwards to form a folded arrangement;

FIG. 6b shows a not-to-scale partial top view of the side of the back section of a hygiene article facing the body, with two accordion-folded material segments, folded on themselves and folded inwards to form a folded arrangement;

FIG. 7 shows an enlarged representation of a material segment folded on itself; and

FIG. 8 shows a schematic sectional view of a material segment folded on itself.

### DETAILED DESCRIPTION

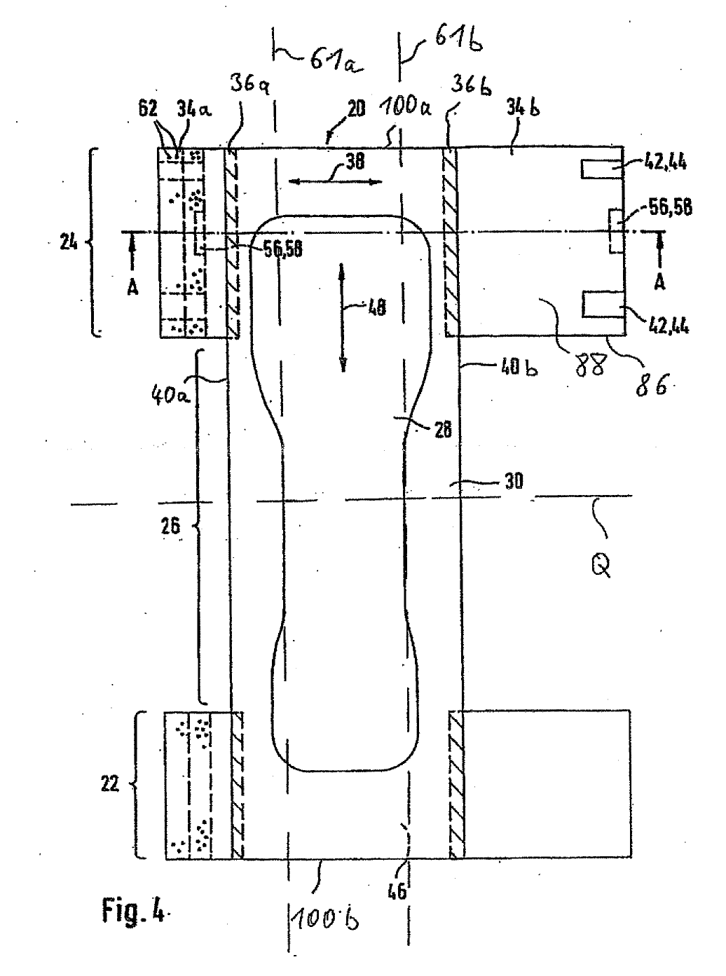

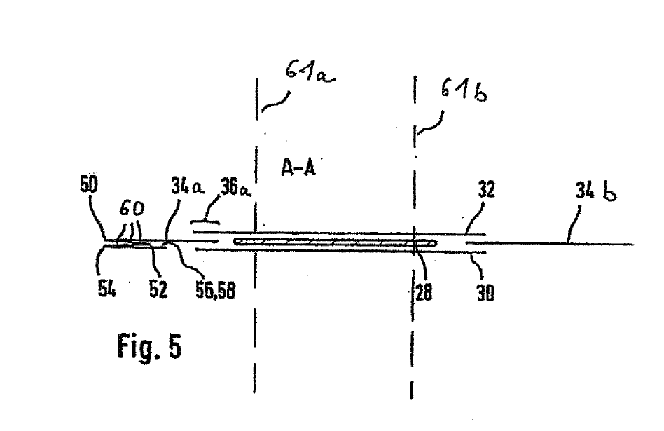

The incontinence article is shown schematically in FIGS. 4 to 8. It comprises a main piece identified overall with the reference numeral 20, also often described as the chassis. The main piece 20 includes a front section 22, a back section 24 and a crotch section 26 lying therebetween which is positioned between the legs of a wearer when the hygiene article is applied to a user. The incontinence article further has a central transverse axis Q which mentally divides the incontinence article into two sections of equal length in the longitudinal direction. The main piece includes an absorbent body 28 which is suitably dimensioned to receive and permanently store bodily fluids. The absorbent body is underpinned by a fluid-impermeable layer 30 which can also form the external safety side of the incontinence article. A fluid-permeable topsheet 32 can be provided above the absorbent body 28 which is shown in the sectioned view of FIG. 5.

In the back section 24, a first material segment 34a forming first side flaps or side sections is attached to a first side edge section 36a of the main piece and a second material segment 34b forming second side flaps or side sections is attached to a second side edge section 36b of the main piece. The material segments 34a and 34b have a rectangular shape. The outer transverse edges of main piece 20 and first material segment 34a and second material segment 34b form the rear edge 100a of the hip opening. Forming side sections on both sides to provide a continuous material segment in the transverse direction 38 which forms both side sections of the incontinence article could also be contemplated.

FIG. 4 shows a view onto the side of the incontinence article facing the body with material segments whose folded arrangement has been loosened and unfolded. The right, second material segment 34b is additionally shown in the drawing in its completely unfolded state. It extends in the transverse direction 38 beyond the longitudinal edge 40b in question of the main piece 20 in the circumferential direction of the hips. The material segment has two closing elements 42 in the form of closing tapes 44 which are folded on themselves and can be unfolded for correct use and which adhere releasably to cooperate with an outside 46 of the front section 22 of the main piece 20 and of the material segments in the front section 22 to close the hygiene article.

Each particular material segment 34a, 34b in the back section of the main piece is, as can be seen from FIG. 4 in the upper left and the sectional drawing from FIG. 5, folded on itself accordion-style along three fold lines 50, 52, 54 extending in the longitudinal direction 48, with partial sections 60 coming to lie on each other.

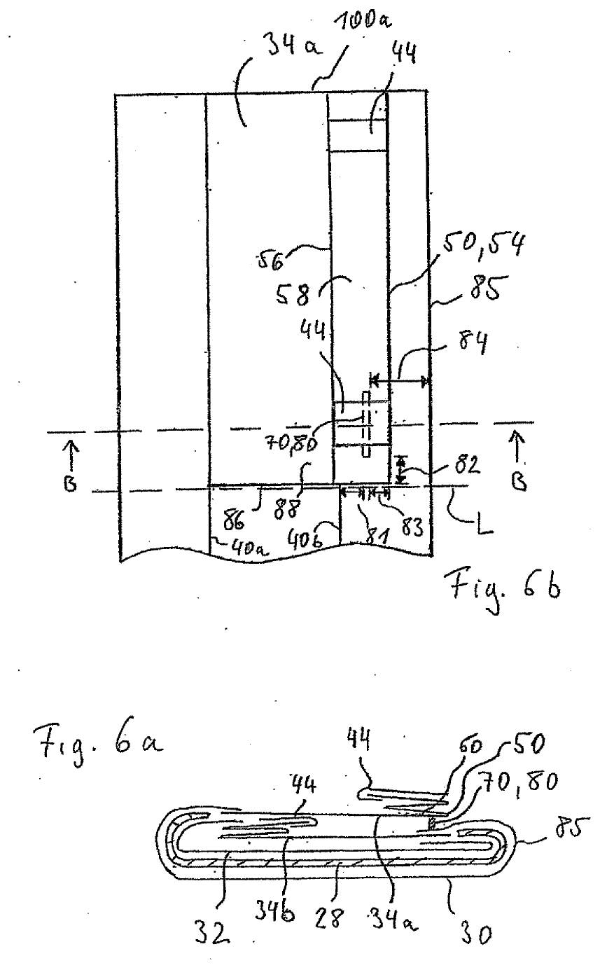

An open longitudinal edge section 56 of the particular material segment 34a, 34b forms a finger grip 58 for grasping the folded material segment 34 to unfold it. When dispensed to the end user, the material segments 34, 34b, based on the drawing from FIG. 5, are folded inwards about fold axes 61a, 61b, which are parallel to the longitudinal direction 48 and run inside the main piece 20 in the embodiment shown, into the position shown in FIG. 6a to form a folded arrangement. In this representation, the second material segment 34b is also shown in an accordion-fold configuration. It can be seen that the second material segment 34b comes to lie under the first material segment 34a. The first material segment 34a is releasably attached at a partial section 60 by means of a first joining point 70 to the fluid-impermeable layer 30 forming the surface of the main piece facing away from the body. FIG. 6b shows a partial top view onto the folded arrangement of the material segments 34a, 34b shown in FIG. 6a. In the interests of clarity, no non-visible components or fold lines are shown with the exception of the first joining point 70. The first joining point 70 is formed by a stripe of hot-melt adhesive extending 40 mm in the longitudinal direction 48 of the incontinence article and 3 mm in the transverse direction of the incontinence article. The distance 81 of the adhesive stripe 80 to the second longitudinal edge 40b of the main piece 20 is 20 mm. The distance 82 of the adhesive stripe 80 from the lower transverse edge 86 of the first material segment 34a is only 20 mm. The adhesive stripe is thus completely within a lower area 88 of the first material segment 34a, in an area of the first material segment 34a which, when viewed in the longitudinal direction 48, is located closer to the lower transverse edge 86 of the first material segment 34a than to the corresponding rear edge of the hip opening 100a. The distance 83 of the adhesive stripe 80 from the fold line 50 of the first material segment bounding the partial section 60 is 15 mm. The distance 84 of the adhesive stripe 80 from the fold edge 85 of the second material segment 34b is 40 mm. It can be seen that the adhesive stripe 80 is located directly below the projected surface formed by the closing tape 44. This has the advantage that the closing means can act almost as an aid for the user to grip in order to release the first joining point 70, in the case shown to release the adhesive stripe 80 and thus to undo the join between the first material segment 34a and the fluid-permeable layer 30.

It can be seen in addition that the specific open longitudinal edge section 56 and thus the finger grip of the material segments 34a, 34b is turned away from a longitudinal center axis of the incontinence article towards the outside so that with a hygiene article spread out on a flat surface, the left hand of a user can conveniently grasp the finger grip 58 of the first material segment 34a from the left and, after loosening the first joining point 70, the right hand of the user can grasp a correspondingly positioned finger grip of the second material segment 34b from the right.

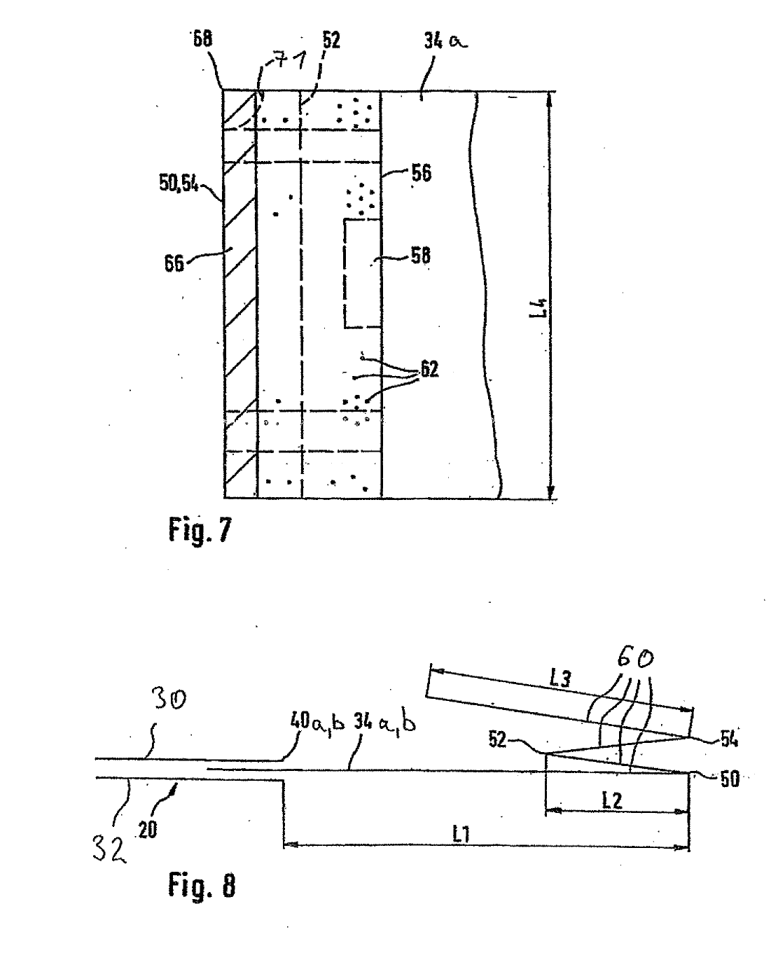

The partial sections 60 folded over each other have no second joining points in a first embodiment. In the embodiment shown here, the folded partial sections 60 of the material segments 34a, 34b on the other hand are releasably attached to each other in the folded configuration by punctiform joining points 62 created by ultrasonic welding, having a diameter of 0.35 mm and an area of 0.0962 mm², which are indicated in FIG. 4. It has been shown that this releasable attachment can be designed such that the specific material segment 34a, 34b can be unfolded completely by a single pull on the particular finger grip 58, with all second joint locations 62 being released or separated. It proves advantageous if no second joint location is provided for the partial sections 60 at a distance of at least 5 mm, preferably of at least 8 mm and further preferably of at least 11 mm from the fold lines 50, 54. This joining point-free area is identified in FIG. 7 by the reference numeral 66. Starting from a point 68 on the fold line 50, 54 farthest removed from the finger grip area 58, no second joining point is provided in a radius 71 indicated schematically. These areas, far removed from the finger grip 58 and located in the proximity of a fold line, are particularly critical with respect to complete unfolding, i.e. a separation of all the second joint locations. It thus proves to be advantageous if only a few, or no, second joining points are provided in these critical areas so that the folded material segments can be separated, or unfolded, with one pull, without multiple repetitions or without having to jerk several times on the finger grip 58.

Using FIG. 8, the dimensions of the partial sections 60 of the individual material segments 34a, 34b become clear. The extent L1 in the transverse direction 38 from each longitudinal edge 40a, 40b of the main piece 20 to fold line 50 is 90 mm. The extent L2 between the fold lines 50-52-54 is 35 mm in each case, and the extent L3 to the open end is 65 mm. The total extension in the transverse direction of the unfolded material segment is therefore 225 mm in the transverse direction 38. The longitudinal extent L4 is 260 mm (FIG. 7).

As can be seen from FIG. 4, sections folded on themselves with a longitudinal extent L4 of only 200 mm and a transverse extent of 225 mm are provided in the front section 22 of the hygiene article material which do not have any closing elements which stiffen the material segments and thus contribute to detaching adjacently located second joining points because of the stiffness of the latter. Second joining points or areas can be provided In the immediate proximity of such stiffening closing elements which are easily separable because of the stiffening.

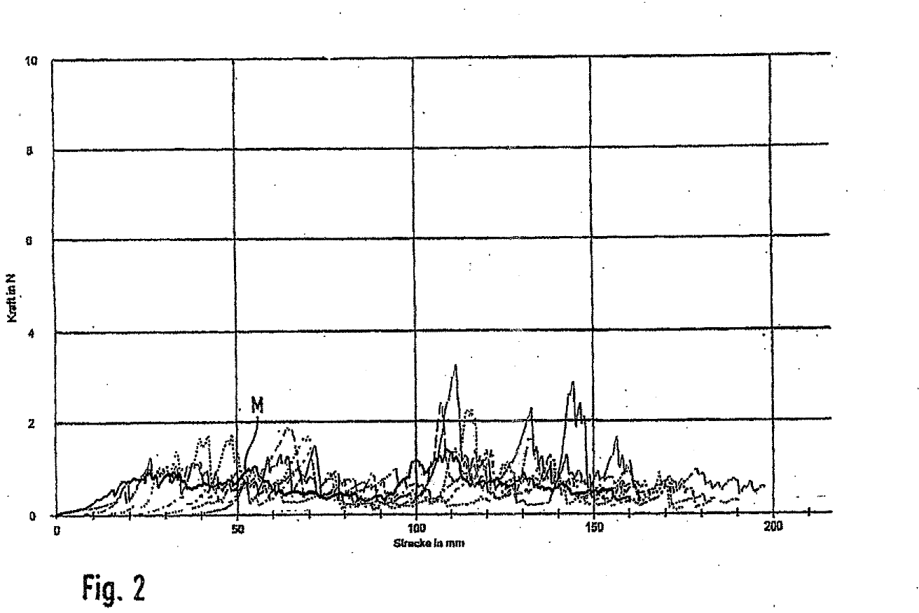

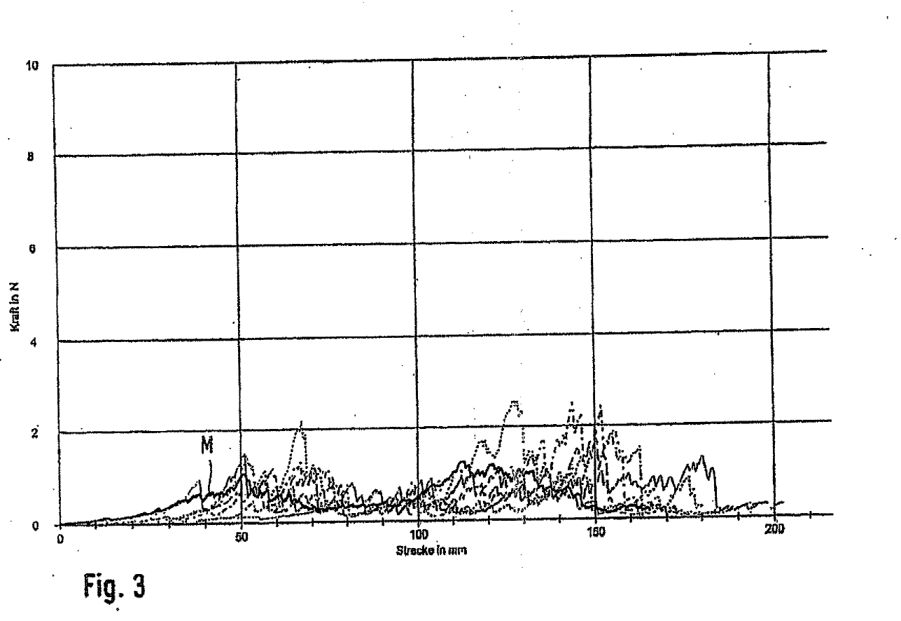

As already mentioned, the measurement results from Table 1 and FIG. 2 were determined in procedures for unfolding the material segments 34a, 34b in the back section 24 and the measurement results from Table 2 and FIG. 3 were determined in procedures for unfolding the material segments in the front section. The measurement results from Table 3 were determined in procedures for separating the first joint location 70, which is configured as an adhesive stripe 80, of the folded arrangement of the material segments 34a, 34b of the back section.

The aforementioned releasable attachment of the folded over material segments can be accomplished, for example, with an adhesive, specifically a hot-melt adhesive, and further specifically with a hot-melt adhesive with permanent adhesive strength. Hot-melt adhesive compounds with low tack have proved to be particularly useful. After opening the joining point and during or after the application of the incontinence article to the wearer, this prevents the adhesive from re-attaching itself unchecked to other components of the incontinence article or to the undergarments or the skin of the wearer. Hot-melt adhesives with a maximum viscosity of 3000 mPa s measured at 160° C. in accordance with ASTM D3236 (from 1988), specifically of 2500 mPa s maximum, further specifically of 2000 mPa s maximum and further specifically of at least 1000 mPa s have proven particularly advantageous. Hot-melt adhesives which contain a polyolefin-copolymer and a polyisobutylene as well as a tackifier prove to be particularly expedient. In addition, these hot-melt adhesives can contain a wax as a plasticizer. A suitable hot-melt adhesive can be obtained, for example, under the name Technomelt HM5432 from Henkel KGaA, Henkelstraβe 67, D-40191 Düsseldorf or under the designation H 5116 from Bostik Nederland B.V., Zeggerfeld 10, NL-4705 RP Roosendahl.

In an alternative aspect, the aforementioned releasable attachment can be accomplished by cold embossing, or by embossing using temperature (thermowelding), or by needling, specifically hot needling, or by ultrasonic or laser welding, or by similar equally effective joining methods.

It proves useful if a partial section of the first material segment is releasably attached to the first joining point or to first jointing points on a partial section of the second material segment. Since first and second material segments are normally formed of identical materials, the choice of the joining method is made easier.

It may prove to be equally useful if a partial section of the first material segment is releasably attached to the surface of the main piece facing away from the body at the first joining point or first joining points, specifically in the immediate vicinity of the first longitudinal side edge of the main piece, which may be of particular usefulness if the fold axis running in the longitudinal direction runs at least in sections inside the main piece. In the immediate vicinity means that the first joining point or the first joining points are distanced from the first longitudinal side edge of the main piece specifically at the most 7 cm, further specifically at the most 4 cm, and further specifically at the most 3 cm. This can prevent the joining point or the joining points negatively affecting the function of the second material segment. For example, in the event an adhesive is used, the risk of adhesive penetrating a porous material comprising the second material segment can be avoided. It furthermore proves advantageous to implement the first joining point or joining points in a lower area of the first material segment. Within the scope of this article, a lower area of the first material segment is understood to mean an area which, when viewed in the longitudinal direction of the incontinence article, is located closer to the lower transverse edge of the first material segment than to the respective upper transverse edge of the first material segment forming one part of the hip opening edge of the incontinence article.

The first joining point or the first joining points may be one or several punctiform adhesive locations. In one aspect, the first joining point includes at least one continuous or discontinuous adhesive stripe, specifically a hot-melt adhesive stripe where the adhesive stripe can extend in the longitudinal direction over the entire dimension of the first material segment, meaning over its entire length. The length of the adhesive stripe can be 10-120 mm, specifically 20-80 mm and further specifically 30-60 mm. The width of the adhesive stripe, meaning its extent in the transverse direction, can be 1-20 mm, specifically 1.5-15 mm and further specifically 2-10 mm.

In further developing the article, it proves advantageous if the first and second material segments, specifically those of the rear section, and even further specifically those of the back section and of the front section, are attached to the main piece of the diaper as separate components. Through the use of different materials in the main piece of the diaper and in first and second material segments, the diaper can be configured with zones of different properties, such as in particular breathability or air permeability, or strength, or softness, or elasticity.

In further developing the article, it can be useful if the material segments in turn are folded on themselves along at least one fold line running in the longitudinal direction. In this, it is useful if partial sections of the material segments folded on themselves and making surface contact over a broad area are releasably attached in this folded configuration at second joining points. This ensures that the material segments are still in an orderly configuration when the first joining point or the first joining points keeping the arrangement folded on itself was detached. In addition, folding the material segments inward during production of the diaper is made easier.

In further developing the article, a finger grip for unfolding the material segment is provided on a partial section of a specific material segment and folded in this way which forms the open end of the material segment in the transverse direction. In the simplest case, this finger grip can be formed by a longitudinal side edge section which the wearer can grasp with his fingers. It would also be conceivable that a separate manually graspable finger grip is provided on the pertinent partial section, which would, however, involve additional production complexity.

Furthermore, it proves useful for handling that the partial section in question does not have any second joining points in the finger grip area, that it is not secured or joined there to additional partial sections or to the main piece in a manner that would impede a wearer's fingers from taking hold or make this more difficult. It would, however, also be conceivable that the wearer inserts his fingers between or through individual second joining points to take hold of the finger grip and then, while pulling, unfold the material segment by releasing the second joining points.

In accordance with another aspect of the incontinence article, it proves useful that the releasable attachment at all the second joining points can be separated when unfolding by a single pull on the finger grip of the specific material segments. This further simplifies handling, and the incontinence article becomes even more user friendly, in particular, regarding use in the case of persons requiring intensive personal care.

The aforementioned complete unfolding of the folded material segments, which can be accomplished by pulling once on a finger grip, that is, by a single pulling motion, means that the user does not have to pull abruptly several times or even tug on a particular material segment until all the joining points between the partial sections of the material segments and, if need be, to the main piece of the incontinence article, are detached.

In another aspect of the article, both the releasable attachment at all the first joining points of the folded arrangement and also the releasable attachment at all the second joining points of the first material segment can be separated when unfolding by a single pull on one finger grip of the first material segment.

In the simplest case, an individual material segment is folded on itself along a fold line so that two partial sections lie on top of each other or abut each other. The material segment is folded on itself along at least two fold lines so that in cross-section a Z-shaped configuration is created. In accordance with one aspect, the material segments are folded on themselves along three fold lines. In accordance with another aspect, the material segments are folded on themselves along four fold lines.

In accordance with another aspect of the incontinence article, the individual finger grips are turned outward in the transverse direction before the material segments are unfolded, that is, turned away from each other and from a longitudinal center axis of the main piece of the diaper when spread out on a flat surface so that they can be grasped comfortably from the left with the user's left hand and from the right with the right hand.

The releasable attachment between the partial sections folded on themselves and possibly also the attachment to the main piece can be formed by several essentially punctiform second joining points. A punctiform joining point of the type mentioned previously means that the joining point has an area (projected onto the X-Y plane of the main piece) of less than 5 mm², specifically of less than 2 mm² and further specifically of less than 1 mm². The second joining points do not have to be strictly punctiform or circular. Shapes departing from punctiform or circular, such as triangular, rectangular, polygonal or oval, are also conceivable and advantageous. The releasable attachment between the folded partial sections of the material segments can be formed by punctiform second joining points created thermally or by ultrasound.

It was recognized as part of the article that the number, distribution or relative surface area of the second joining points or the adhesive strength of the releasably attached partial sections can be selected in such a way that the releasable attachment at all the second joining points can be separated when unfolding by a single pull on the specific finger grip of the material segments. This can be assisted in an advantageous manner by reducing the number or the relative surface area of the second joining points or the adhesive strength of the releasably attached partial sections with the distance from the finger grip of the material segment. It was recognized that the further an area of the folded partial sections of the material segments is removed from the finger grip, the less the strength of the bond between the partial sections should be in order to detach all the second joining points or areas by a single pull on the particular finger grip of the material segments, that is, by a single unfolding motion. Consequently, it was also recognized that there is no difficulty in making the releasable attachment of the folded partial sections sufficiently strong in the proximity of the finger grip to meet requirements. In this way reliable transportation of the flat material webs, which have been folded before or in the high-speed diaper manufacturing machine, is ensured without material segments projecting laterally from the main piece of the incontinence article fluttering or folded partial sections being displaced inside the fold. A neat appearance also results later during the folding of the complete product, particularly with the provision of the folded arrangement of the material segments.

In a further aspect, it proves advantageous if, within a radius of 1.5 cm from a point on the furthest removed fold line, the point being furthest removed in the planar direction from the finger grip of the folded configuration, the abutting partial sections are not joined by second joining points. This furthest removed point will be a point on the fold line lying at the open edge of the material segments. However, it proves advantageous it over the entire extent of the fold line under consideration, specifically farthest away from the finger grip, the abutting partial sections are not joined together at a distance of 5 nm, specifically of 8 mm and further specifically of 10 mm removed from this fold line.

It proves further useful if the planar extension of the partial sections, folded on themselves and abutting each other, can be (mentally) divided into two approximately equal halves by a straight line running in the longitudinal direction and if the number, or the relative surface area of the two joining points or areas, or the adhesive strength of the releasably attached partial sections is different in these two halves. It proves to be particularly useful if the number, or the relative surface area of the second joining points, or the adhesive strength of the releasably attached partial sections in the half facing the finger grip in the transverse direction, that is, the adjoining half, is greater than in the half facing away from the finger grip in the transverse direction.

The material segments attached to the main piece of the incontinence article are, as already mentioned at the beginning, configured wider (perpendicular to the direction around the hips) than is the case with typical belt diapers. The width, that is, the extension of a material segment in the longitudinal direction of the hygiene article, is preferably at least 10 cm in the area where it is attached to the main piece, specifically at least 14 cm, in particular specifically 18 cm, and further specifically 22 cm.

The extension of a material segment joined to the main piece when unfolded is at least 10 cm, specifically at least 15 cm, and further specifically at least 18 cm in the transverse direction beyond the longitudinal edge of the main piece, which is equivalent to the circumference around the hips when the article is worn. It can be 35 cm at the most, specifically 30 cm at the most and further specifically 27 cm at the most. It is also conceivable and advantageous for individual incontinence articles if such material segments projecting from the main piece in the transverse direction are attached both in the front section as well as in the back section. In this case, it proves advantageous if all such material segments of the incontinence article are folded within the meaning of the invention and releasably attached.

In order to close the incontinence article in the position when worn by a user, the material segments have closing elements which can be configured to be mechanically adhering or adhesive and which for their part are arranged in a folded configuration on the material segments, and can be unfolded for use. It proves expedient if the material segments in the back section have such closing elements which can cooperate with a landing zone on the main diaper piece and/or on the front material segments. The closing elements can attach releasably or be adhesive.

The material segments attached to the main piece can be formed from a non-woven material, specifically spunbond materials (S), or spunbond melt blown materials (SM), or melt blown layers (SMS) provided on both sides with spunbond materials, or also carded non-woven materials can be used. Non-woven laminates, in particular dual-layer, triple-layer or multi-layer combinations of the aforementioned non-woven materials can be used. The joining of the individual layers can be accomplished by customary and known methods, for example by thermal joining processes (welding, specifically laser welding, hot-melt, air-through), or by ultrasonic welding processes; cold pressing, needling, sewing or bonding of non-woven materials can also be contemplated. Joining using textile woven fabrics or knit fabrics, that is, using materials exhibiting a textile bond in the broadest sense can also be contemplated. The material segments joined laterally to the main piece are configured to breathe, with microporosity in particular being considered advantageous, permitting both an exchange of air and permeability for moisture in the form of vapor. The material segments advantageously have a basis weight of 10 to 150 g/m², specifically 20-100 g/m², further specifically 25-50 g/m².

According to a further aspect of the article, the material segments attached to the main piece can also be configured in such a way that they are less rigid than the main piece or the materials of the main piece forming the chassis, such as in particular the backsheet or a laminate of the main piece consisting of backsheet and topsheet. A skin-friendly side section of the hygiene article can be achieved in this way which preferably feels like a textile or a non-woven material and is experienced as pleasant by the wearer.

It proves particularly useful according to an independent inventive concept that when the material segments are unfolded by pulling on the particular finger grip, the second joining point or the second joining points offer a peak resistance averaged over the unfolding process of 2.5 N maximum, specifically of 2.4 N maximum, specifically of 2.3 N maximum, specifically of 2.2 N maximum, specifically of 2.1 N maximum, and further specifically of 2.0 N maximum. If the tensile force at each moment is determined when pulling on the particular finger grip during an unfolding process, and if those force spikes which differ from immediately adjacent areas by at least 0.5 N are taken into account, it is possible to average all the force spikes determined in this way during an unfolding process and so calculate a peak force averaged over the number of these force spikes.

If not just one unfolding procedure is taken into account, but six unfolding procedures for six identically produced and folded material segments and the peak forces are averaged over the six unfolding procedures as previously determined, they are preferably 2.3 N at the most, specifically 2.0 N at the most, specifically 1.8 N at the most, specifically 1.7 N at the most, specifically 1.6 N at the most, and further specifically 1.5 N at the most.

It further proves particularly advantageous that in accordance with a further intrinsically independent concept, the material segments are folded and pre-attached by second joining points in such a way that the effort required when unfolding a material segment with one pull, averaged over six unfolding events is at the most 120 Nmm, specifically 115 Nmm at the most, specifically 110 Nmm at the most specifically 105 Nmm at the most, specifically 100 Nell at the most, specifically 95 Nmm at the most, and specifically 90 Nmm at the most.

In what follows, a test is described to determine the forces to be overcome when unfolding and thus to detach the second joining point or second joining points of the material segments folded on themselves and to test the unfolding of the material segment in one pull. The force occurring at each moment across the opening distance is determined and recorded using tensile test equipment in accordance with EN ISO 527-1 (April 1996).

Test Preparation:

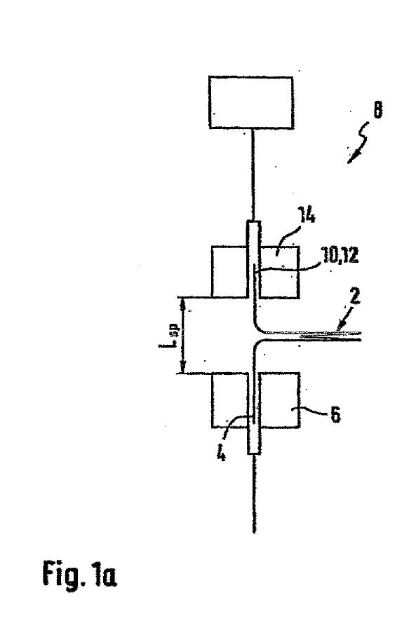

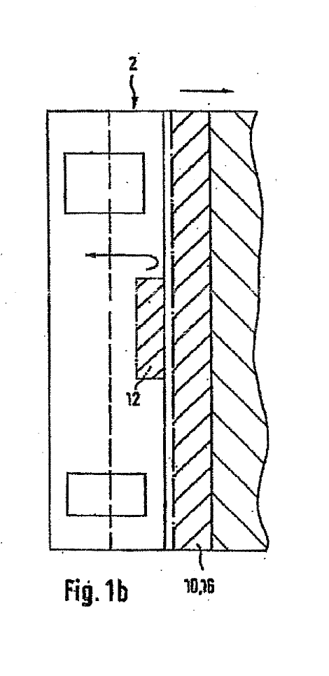

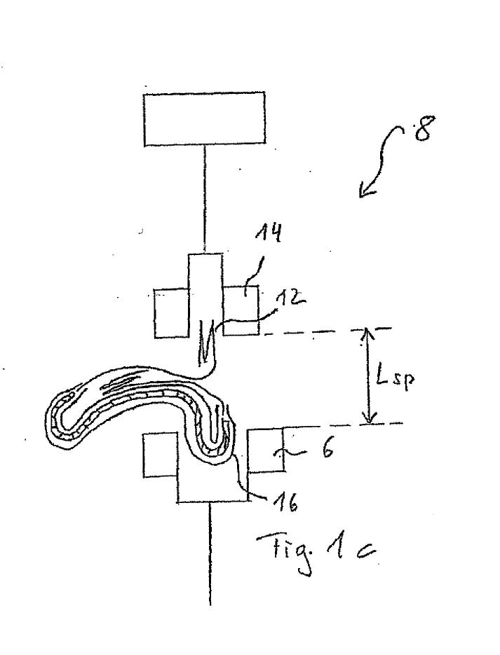

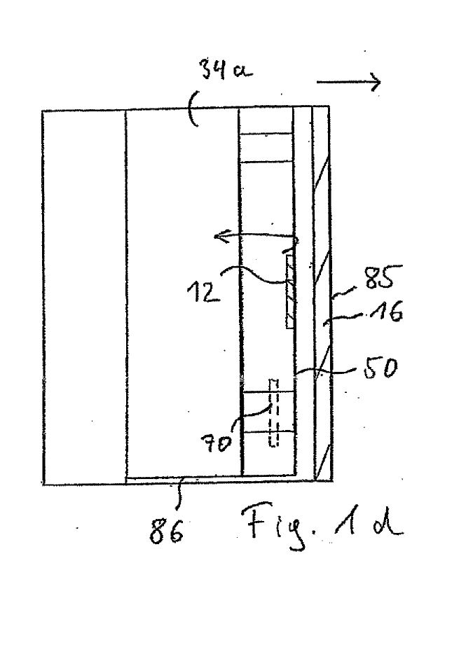

In the case of a hygiene article, the inward folded arrangement of the material segments is first unfolded by releasing the first joining points. Then a material segment joined to the main piece and folded on itself along a longitudinal side edge of the main piece is separated from the hygiene article by destroying the join. A blade or scissors can be used for this. The material segment 2 forming the relevant side part is tightly clamped according to FIGS. 1a and 1b with a longitudinal side edge 4 to a lower clamp 6 of the tensile test equipment along its entire length (in the longitudinal direction of the incontinence article) with which it was previously attached to the main piece. The lower clamp 6 of the tensile test equipment 8 must therefore be of a correspondingly length, in practical terms a length of 300 mm. On the opposite open longitudinal side edge 10 of the separated material segment 2, which forms a finger grip 12, the moveable clamp 14 of the tensile test equipment 8 is clamped over a length of 60 mm. When the longitudinal extension of the material segment is less than 160 mm, the material segment 2 is attached to the moveable clamp 14 at its free longitudinal side edge 10 over a length of 30 mm. FIG. 1b, which is not to scale, shows the material segment 2 in its folded configuration, with the area 16 on the longitudinal side edge 10 which can be fastened in the clamp 6 and finger grip 12 which can be fastened in the clamp 14 shown cross-hatched. The arrows indicate the direction of pull in the tensile test equipment. The clamped length Lₛₚ is also indicated in FIG. 1a.

A tensile test is conducted by controlled movement of this moveable clamp 14.

Test Parameters:

Test speed of the moveable clamp: 300 min/min

Clamped length: 10 mm

Measurement range: Length of the transverse extension of the unfolded material segment

Preload: 0.01N

Analysis:

The test result of the tensile test is shown as tensile force occurring in the material segment and determined between the clamps rounded to two decimal places in N. A force/displacement plot is generated.

FIG. 2 shows such a force/displacement plot. The results of six tensile tests are shown in FIG. 2 and the heavy line M is an averaged curve for an incontinence article to be described in what follows. The peak forces Fmax and the average values determined for Fmax for a particular unfolding procedure are given in Table 1 below. A force spike of Fmax was assumed if it differed by 0.5 N from an adjacent minimum force. If the force/displacement plot of the detachment procedure were to have only a single force spike, then this applies, Maximum force Fmax Average Fmax.

Table 1 also shows the effort required for opening in Nmm which was determined mathematically from the tensile forces recorded and the displacement.

TABLE 1

| | | | W [displacement] in |
|---|---|---|---|
| Number | Average Fₘₐₓ in N | Fₘₐₓ in N | Nmm |
| 1 | 1.78 | 3.24 | 95.69 |
| 2 | 1.41 | 2.41 | 91.63 |
| 3 | 1.61 | 2.25 | 89.91 |
| 4 | 1.16 | 1.39 | 62.66 |
| 5 | 1.12 | 1.62 | 60.84 |
| 6 | 1.69 | 2.86 | 95.40 |
| X(n = 6) | 1.46 | 2.29 | 82.69 |

The results from FIG. 2 and Table 1 were determined during the unfolding of material segments which were used in a back section of an inventive hygiene article which will be described in what follows, with these material segments at the same time having stiffening closing elements.

Equivalent measurements were conducted on material segments provided in a front section of the hygiene article with a lesser longitudinal extension and without stiffening closing elements. The results are shown in FIG. 3 and in Table 2 below in the corresponding form.

TABLE 2

| | | | W [displacement] in |
|---|---|---|---|
| Number | Average Fₘₐₓ in N | Fₘₐₓ in N | Nmm |
| 1 | 1.05 | 1.48 | 64.67 |
| 2 | 1.20 | 1.77 | 73.73 |
| 3 | 1.52 | 2.54 | 105.45 |
| 4 | 1.73 | 2.49 | 93.93 |
| 5 | 1.32 | 1.88 | 59.04 |
| 6 | 1.10 | 1.30 | 66.19 |
| X(n = 6) | 1.32 | 1.91 | 77.17 |

In accordance with an intrinsically independent feature, it proves advantageous that, when separating the arrangement folded on itself, the first joining point or the first joining points offers a resistance averaged by way of the detachment procedure of 3.0 N at the most, specifically 2.0 N at the most, specifically 1.5 N at the most and further specifically of 1.0 N at the most.

If not just one detachment procedure, but six detachment procedures of six identically shaped and folded material segments are taken into account and the peak forces are averaged over the six detachment procedures just as previously determined, they are at the most 2.5 N, specifically at the most 2.0 N, specifically at the most 1.5 N and further specifically at the most 1.0 N.

It furthermore proves useful that in accordance with an intrinsically independent feature, the material segments in the folded arrangement are pre-attached in such a way by a first joining point or by first joining points that the effort required when detaching the first joining point or first joining points with one pull, averaged over six detachment procedures, is 50 Nmm at the most, specifically 40 Nmm at the most, specifically 30 Nmm at the most, and further specifically 20 Nmm at the most.

By limiting the detaching forces and the effort required for detachment, it is ensured that the folded arrangement can be detached by the user without difficulty, in particular without damaging the specific material components.

In what follows, a test is described to determine the forces to be overcome when detaching the first joining point or first joining points attaching the arrangement folded on itself. The force at each moment over the opening distance is determined and recorded using a tensile test machine in accordance with EN ISO 527-1 (April 1996).

Test Preparation;

First, with a hygiene article having an arrangement of material segments folded on itself shown in FIGS. 6a and 6b, the entire front or rear section 22, 24 having this arrangement folded on itself is separated by a cut along a line L in the transverse direction 38 of the hygiene article, preferably immediately below the lower transverse edge 86 of the first material segment 34a, but in any case below the first joining point 70. A blade or scissors can be used to do this.

The front or rear section in question, as shown in FIGS. 1c and 1d, is then firmly clamped in the center at the folding edge 85 at a lower clamp 6 of the tensile test apparatus over an area 16 comprising its entire length (in the longitudinal direction of the incontinence article). The lower clamp 6 of the tensile test apparatus 8 must consequently be of a corresponding length, advantageously a length of 300 mm. At the fold line 50, which bounds the partial section 60 to which the first material segment 34a is joined at the first joining point 70, the moveable clamp 14 of the tensile test apparatus 8 is securely clamped in the center over a length of 60 mm and a depth of 5 mm in an area designated with reference numeral 12. FIG. 1, which is not to scale, shows the back section in the folded arrangement of the material segments, with the area 16 on the longitudinal side edge 10 which can be fastened in the clamp 6 and finger grip area 12 which can be fastened in the clamp 14, shown cross-hatched. The arrows indicate the direction of pull in the tensile test equipment, In FIG. 1c, in which the first joining point 70 cannot be seen and is therefore not shown, the clamped length Lₛₚ is also indicated.

A tensile test is conducted by controlled movement of this moveable clamp 14.

Test Parameters:

Test speed of the moveable clamp: 300 mm/min

Clamping length: 50 mm

Measurement range: until complete separation of the first joining point or first joining points of the folded arrangement

Preload: 0.2 N

Analysis:

The test result of the tensile test is given as tensile force in N occurring in the front or rear section and between the clamps rounded to two decimal places. A force/displacement plot is generated.

As with the previously described test method to determine the opening force for a material segment folded on itself the peak forces Fmax and the averages of Fmax for a particular separating procedure are given. The starting point was a force peak of Fmax if it differed from an adjacent force minimum by 0.1 N. If the force/displacement plot of the detachment process were to show only a single force spike, this applies: Peak force Fmax=average Fmax. Table 3 below additionally shows the effort required for detachment in Nmm which was determined mathematically from the tensile forces recorded and the displacement

| | | | W [displacement] in |
|---|---|---|---|
| Number | Average Fₘₐₓ in N | Fₘₐₓ in N | Nmm |
| 1 | 1.00 | 1.00 | 24 |
| 2 | 0.93 | 0.93 | 15 |
| 3 | 0.95 | 0.95 | 19 |
| 4 | 0.81 | 0.81 | 15 |
| 5 | 0.85 | 0.91 | 20 |
| 6 | 0.91 | 0.91 | 19 |
| X(n = 6) | 0.91 | 0.92 | 18.7 |

1. An absorbent incontinence article having a main piece including a front section, a back section and a crotch section between the front section and the back section lying in the a longitudinal direction which is adapted to be positioned between the legs of a user, the main piece comprising:
an absorbent body;
first material segments adjoining one of the back section and the front section which extend in a transverse direction beyond first longitudinal side edges of the main piece;
second material segments adjoining one of the back section and the front section which extend in the transverse direction beyond second longitudinal edges of the main part, where;
the first and second material segments connecting the front section and the back section to each other when the article is worn; and wherein
before the folded article is used, the first and second material segments are folded inward about a fold axis running in the longitudinal direction onto the side of the main part facing the body to form an arrangement folded on itself such that the second material segment lies at least partially under the first material segment, and this the folded arrangement is releasably attached at a first joining point.

2. The incontinence article from claim 1, wherein a partial section of the first material segment is releasably attached at a first joining point to a partial section of the second material segment.

3. The incontinence article from claim 1, wherein the fold axis runs inside the main piece

4. The incontinence article from claim 1, wherein a partial section of the first material segment is releasably attached at a first joining point to the surface of the main piece facing away from the body in the immediate proximity of the second longitudinal edge of the main piece.

5. The incontinence article from claim 2, wherein the first joining point is formed by at least one single joining point.

6. The incontinence article claim 2, wherein the first joining point of the folded arrangement is formed by one of thermally and ultrasound joining points.

7. The incontinence article from claim 2, wherein the first joining point of the folded arrangement is formed by an adhesive.

8. The incontinence article from claim 2, wherein the first joining point includes an adhesive stripe, the adhesive stripe substantially extending about 10 to about 120 mm specifically 20-80 mm and further in the transverse direction.

9. The incontinence article from claim 1, wherein the material segments (34a, 34b) are attached as separate elements to the main piece.

10. The incontinence article from claim 1, wherein each of the material segments is folded on itself along at least one fold line running in the longitudinal direction, whereby partial sections of the individual material segments come to lie on each other.

11. The incontinence article from claim 10, wherein the partial sections of the material segments folded on each other and making surface contact over a broad area are releasably attached in is the folded configuration at a second joining point.

12. The incontinence article from claim 11, wherein the second joining point is formed by at least a single second joining point.

13. The incontinence article from at least one of the claims 10-12, claim 11, wherein a finger grip for unfolding the material segment is provided at the partial section of a folded material segment which forms an open end of the material segment in the transverse directions.

14. The incontinence article from at least one of the claim 13, wherein the releasable attachment at the second of the material segments.

15. The incontinence article from claim 13, wherein both the releasable attachment at the first joining point of the folded arrangement and the releasable attachment at the second joining points of the first material segment can be separated when unfolding by a single pull on an individual finger grip of the first material segment.

16. The incontinence article from claim 11, wherein the second joining point of the folded partial sections of the material segments is formed by one of a thermal and ultrasound second joining points.

17. The incontinence article from claim 13, wherein the partial section in the area of the finger grip has no second joining point.

18. The incontinence article from claim 1, wherein the material segments are rectangular in shape.

19. The incontinence article from claim 10, wherein a folded material segment is folded on itself along at least two fold lines.

20. The incontinence article from claim 19, wherein a folded material segment is folded on itself along one of three and four fold lines (50, 52, 54).

21. The incontinence article from claim 13, wherein, before it is used, the finger grips are turned outward away from each other in the transverse direction so that a left hand of a user can conveniently grasp the finger grips from the left and a right hand from the right.

22. The incontinence article from claim 11, characterized by an area of the material segments in a folded configuration in which one of the number and the relative surface area of the second joining point and the adhesion strength of the releasably attached partial sections of the material segments decreases with the distance from the area of the finger grip in the longitudinal direction.

23. The incontinence article from claim 11, characterized by an area of the material segments in a folded configuration in which one of the number or and the relative surface area of the second joining points or and the adhesion strength of the releasably attached partial sections of the material segments decreases with the distance from the area of the finger grip in the transverse direction.

24. The incontinence article from claim 11, wherein the abutting partial sections of a material segment are not joined together in a radius of about 1.5 cm from a point farthest removed from the finger grip on the farthest removed fold line.

25. The incontinence article from claim 11, wherein the partial sections of a particular material segment are not joined together in a space of about 5 mm to about 10 mm from the fold line, farthest removed from the finger grip.

26. The incontinence article from claim 11, wherein the surface extension of the partial sections folded over each other and abutting each other are divided into two approximately equal halves by a straight line running in the longitudinal direction; and wherein
at least one of the number and the relative surface area of the second joining points and the adhesive strength of the releasably attached partial sections of a particular material segment is different in the two halves.

27. The incontinence article from claim 26, wherein one of the number and the relative surface area of the second joining point and the adhesive strength of the releasably attached partial sections the material segment is greater in the half facing toward the finger grip in the transverse direction than in the half facing away from the finger grip in the transverse direction.

28. The incontinence article from claim 1, wherein an extension of an attached material segment in the longitudinal direction in the area of the attachment to the main piece is at least 10 cm to at least 22 cm.

29. The incontinence article from claim 1, wherein an extension of an attached material segment in the unfolded state in the transverse direction beyond the longitudinal edge of the main piece is at least 10 cm to at least 22 cm.

30. The incontinence article from claim 1, wherein the material segments have closing elements which are located on the material segments, in a folded configuration which can be unfolded for use.

31. The incontinence article from claim 1, wherein the material segments attached to the main piece are formed of a non-woven material.

32. The incontinence article from one or more of the preceding claims claim 1, wherein the material segments attached to the main piece are less stiff than the main piece.

33. The incontinence article from claim 13, wherein the second joining point offers resistance with a peak force averaged over the unfolding procedure of 2.5 N maximum, specifically 2.4 N maximum, specifically of 2.3 N to 2.0 N maximum when the material segments are unfolded by pulling on the finger grip.

34. The incontinence article from claim 13, wherein the second joining point offers resistance with a peak force averaged over six unfolding procedures of 2.3 N to 1.5 N when the material segments are unfolded by pulling on the finger grip.

35. The incontinence article from claim 11, wherein the effort averaged over six unfolding procedures is 120 Nmm to 90 Nmm when releasing the second joining point with one pull.

36. The incontinence article from claim 2, wherein the first joining point offers resistance with a peak force averaged over the separation procedure of about 3.0 N maximum, specifically of 2.0 N to about 1.0 N when loosening the folded arrangement.

37. The incontinence article from claim 2, wherein the first joining point offers resistance with a peak force averaged over the six detaching procedures of 3.0 N to 1.0 N maximum when loosening the folded arrangement.

38. The incontinence article from one or more of the aforementioned claims claim 2, wherein the effort required averaged over six detaching procedures is 50 Nmm maximum, specifically 40 Nmm maximum, specifically 30 Nmm maximum and further specifically to 20 Nmm maximum when loosening the first joining point or first joining points (70) with one pull.

39. The incontinence article from claim 2, wherein the first joining point located in a lower area of the first material segment.

40. A method for manufacturing an incontinence article in accordance with claim 1 comprising the steps of:
a. feedings a main diaper piece in a longitudinal direction with first material segments a bordering one of the rear section and the front section which extend in a transverse direction beyond first lateral longitudinal edges of the main piece and with second material segments bordering one of the rear section or the front section which extend in the transverse direction beyond second lateral longitudinal edges of the main piece, and folding the first and second material segments on themselves at least along a first fold line running in the longitudinal direction;
b. folding over of the material segments folded on themselves around a fold axis running in the longitudinal direction inwards onto the side of the main piece facing the body to form an arrangement folded on itself such that the second material segment comes to lie at least in sections underneath the first material segment; and
c. bonding of the folded over arrangement at a first joining point.

41. The method from claim 40 further comprising the step of:
locating the fold axes within the main piece.

42. The method from claim 41, wherein the fold axes run at least in sections through the absorbent body.
